(19) United States
(12) Patent Application Publication
(10) Pub. No.: US 2021/0128801 A1
(43) Pub. Date: May 6, 2021

(54) SYRINGE PUMP

(71) Applicant: DIALITY INC., IRVINE, CA (US)

(72) Inventors: CLAYTON POPPE, IRVINE, CA (US); RONALDO SANTIAGO, NAPERVILLE, IL (US); SCOTT BEU, NAPERVILLE, IL (US)

(73) Assignee: DIALITY INC., IRVINE, CA (US)

(21) Appl. No.: 17/088,421

(22) Filed: Nov. 3, 2020

Related U.S. Application Data
(60) Provisional application No. 62/931,037, filed on Nov. 5, 2019.

(51) Int. Cl. A61M 1/00; A61M 1/16; A61M 1/36

(52) U.S. Cl. CPC A61M 1/0068; A61M 1/1647; A61M 2205/3306; A61M 2205/332; A61M 1/3673

(57) ABSTRACT

A syringe pump to deliver heparin to into the blood circuit of a hemodialysis system. The syringe pump is configured to receive a syringe having a plunger movable within a lumen of an elongate tubular member. The syringe pump may include a housing having a recess configured to receive at least a portion of the syringe, a drive mechanism for moving the plunger within the lumen, the drive mechanism comprising a motor and a lead screw; and a grabber mechanism. The grabber mechanism includes a control arm, back panel, and upper and lower control fingers, the control fingers each have first and second ends, and a curved portion therebetween having a width, an interior edge, and an exterior edge, wherein the first ends of the upper and lower control fingers are coupled to the control arm via first and second spring hinges.

### CROSS-REFERENCE TO RELATED APPLICATION

This application claims priority to, and the benefit of, U.S. Provisional Application No. 62/931,037, filed Nov. 5, 2019, which is hereby expressly incorporated by reference in its entirety for all purposes.

### BACKGROUND OF THE INVENTION

The present invention relates to a syringe pump, e.g., for use with a hemodialysis system to deliver heparin.

Applicant hereby incorporates herein by reference any and all patents and published patent applications cited or referred to in this application.

Hemodialysis is a medical procedure that is used to achieve the extracorporeal removal of waste products including creatine, urea, and free water from a patient's blood involving the diffusion of solutes across a semipermeable membrane. Failure to properly remove these waste products can result in renal failure.

During hemodialysis, the patient's blood is removed by an arterial line, treated by a dialysis machine, and returned to the body by a venous line. The dialysis machine includes a dialyzer containing a large number of hollow fibers forming a semipermeable membrane through which the blood is transported. In addition, the dialysis machine utilizes a dialysate liquid, containing the proper amounts of electrolytes and other essential constituents (such as glucose), that is also pumped through the dialyzer.

Typically, dialysate is prepared by mixing water with appropriate proportions of an acid concentrate and a bicarbonate concentrate. Preferably, the acid and the bicarbonate concentrate are separated until the final mixing right before use in the dialyzer as the calcium and magnesium in the acid concentrate will precipitate out when in contact with the high bicarbonate level in the bicarbonate concentrate. The dialysate may also include appropriate levels of sodium, potassium, chloride, and glucose.

The dialysis process across the membrane is achieved by a combination of diffusion and convection. The diffusion entails the migration of molecules by random motion from regions of high concentration to regions of low concentration. Meanwhile, convection entails the movement of solute typically in response to a difference in hydrostatic pressure. The fibers forming the semipermeable membrane separate the blood plasma from the dialysate and provide a large surface area for diffusion to take place which allows waste, including urea, potassium and phosphate, to permeate into the dialysate while preventing the transfer of larger molecules such as blood cells, polypeptides, and certain proteins into the dialysate. Typically, the dialysate flows in the opposite direction to blood flow in the extracorporeal circuit. The countercurrent flow maintains the concentration gradient across the semipermeable membrane so as to increase the efficiency of the dialysis.

Because hemodialysis requires extracorporeal blood flow, a form of anticoagulation is needed to prevent thrombosis or clotting in the blood circuit. Heparin is commonly injected into the blood circuit to prevent clotting. A standard procedure is to inject a bolus heparin dose at the start of hemodialysis, followed by additional doses mid-treatment to maintain anticoagulation. See https://www.uptodate.com/contents/hemodialysis-anticoagulation#H3683741.

There are numerous risks, however, associated with the use of anticoagulants to prevent clotting in the hemodialysis system. For instance, some patients (such as those suffering from end-stage renal disease (ESRD)), already have an increased risk of bleeding. See Sahota, S. and Rodby, R. “Inpatient hemodialysis without anticoagulation in adults.” CLIN KIDNEY J. 7(60: 552-53 (December 2014). Heparin use increases the risk of hemorrhage, and “can also cause hypertriglyceridemia by reducing endothelium-bound lipoprotein lipase, contribute to hyperkalemia by suppressing aldosterone production in the zona glomerulosa and is associated with immune and non-immune mechanisms that can lead to mild-to-severe thrombocytopenia and with or without thrombosis.” Id. at 553.

Moreover, there are also problems associated with the heparin pump itself. Heparin pumps are known to fail. This can be especially problematic if the patient is dialyzing overnight and is awakened by a loud alarm because, e.g., the dialyzer has clotted. The patient is then forced to trouble-shoot the problem with the dialyzer after being awakened from a sound sleep. Many of the dialysis patients are elderly or suffer from disabilities such as arthritis and have difficulties with the mechanics of loading, unloading, connecting, and disconnecting the heparin pump with one hand.

Accordingly, there is a significant need for a heparin pump that consistently delivers the correct amount of drug for use with hemodialysis systems and is easy to use for the patient.

### SUMMARY OF THE INVENTION

According to a first aspect of the invention, a hemodialysis system is provided including an arterial blood line for connecting to a patient's artery for collecting blood from a patient, a venous blood line for connecting to a patient's vein for returning blood to a patient, a heparin pump, a reusable dialysis machine, and a disposable dialyzer. More details of a hemodialysis system can be found in U.S. application Ser. No. 16/659,941, published as US 2020/0129686, and U.S. application Ser. No. 17/087,383, filed Nov. 2, 2020, which are hereby expressly incorporated by reference in their entirety for all purposes.

The arterial blood line and venous blood line may be typical constructions known to those skilled in the art. For example, the arterial blood line may be traditional flexible hollow tubing connected to a needle for collecting blood from a patient's artery. Similarly, the venous blood line may be a traditional flexible tube and needle for returning blood to a patient's vein. Various constructions and surgical procedures may be employed to gain access to a patient's blood including an intravenous catheter, an arteriovenous fistula, or a synthetic graft.

Preferably, the disposable dialyzer has a construction and design known to those skilled in the art including a blood flow path and a dialysate flow path. The term “flow path” is intended to refer to one or more fluid conduits, also referred to as passageways, for transporting fluids. The conduits may be constructed in any manner as can be determined by ones skilled in the art, such as including flexible medical tubing or non-flexible hollow metal or plastic housings. The blood flow path transports blood in a closed loop system by connecting to the arterial blood line and venous blood line for transporting blood from a patient to the dialyzer and back to the patient. Meanwhile, the dialysate flow path transports dialysate in a closed loop system from a supply of dialysate through a connector to the dialyzer and back through a connector to the dialysate supply. Both the blood flow path and the dialysate flow path pass through the dialyzer, but are separated by the dialyzer's semipermeable membrane.

In one embodiment, the syringe pump is configured to receive a syringe having a plunger movable within a lumen of an elongate tubular member and includes a housing having a recess configured to receive at least a portion of the syringe; a drive mechanism for moving the plunger within the lumen, the drive mechanism comprising a motor and a lead screw; and a grabber mechanism. The grabber mechanism comprises a control arm, a back panel, and upper and lower control fingers. The control arm is coupled to the lead screw. The upper and lower control fingers each have first and second ends, and a curved portion therebetween having a width, an interior edge, and an exterior edge. The first ends of the upper and lower control fingers are coupled to the control arm via first and second spring hinges. At least a portion of each of the curved portions have a beveled surface such that the widths of the curved portions are smaller at the interior edges than at the exterior edges. The gap(s) may be located between the back panel and an opposite side of the beveled surfaces of the upper and lower control fingers.

In another embodiment, the drive mechanism can also include an elliptical tube having a first end, a second end, a lumen therebetween, and a female threaded element configured to receive lead screw. The elliptical tube may prevent rotation of the drive mechanism. The control arm further comprises an elongate elliptical extension and wherein the elliptical tube is coupled to the elongate elliptical extension and at least a portion of the lead screw is disposed within the lumen of the elliptical tube.

In another embodiment, the grabber mechanism further comprises a force sensor. The grabber mechanism may further include a pressure plate located adjacent the force sensor. The pressure plate may be configured to contact the force sensor and an enlarged end of a pusher of a syringe. The force sensor may be a flexible resistive force sensor. The force sensor may be capable of detecting an occlusion in a conduit connected to a syringe received in the syringe pump. Additionally or in the alternative, the force sensor may be capable of detecting a presence of a plunger in the grabber mechanism.

In another embodiment, the syringe pump may further include an optical sensor. The optical sensor may be located behind the syringe, e.g., in the housing. The optical sensor may detect the presence of a syringe in the recess of the housing.

In another embodiment, the drive mechanism further comprises an encoder. The encoder may be configured to verify that the motor is turning the lead screw at a set rate. The encoder may be a linear encoder or a rotary encoder.

### BRIEF DESCRIPTION OF THE DRAWINGS

FIG. 1 is a perspective top view of one embodiment of a heparin pump according to the invention.

FIG. 2 is a side view of one embodiment of a heparin pump according to the invention.

FIG. 3 is a side view of one embodiment of a heparin pump according to the invention.

FIG. 4 is a is a perspective bottom view of one embodiment of a heparin pump according to the invention.

FIG. 5A is a top view of one embodiment of a grabber mechanism according to the invention.

FIG. 5B is a perspective view of one embodiment of a grabber mechanism according to the invention.

FIG. 5C is a side view of one embodiment of a grabber mechanism according to the invention.

FIG. 5D is an end view of one embodiment of a grabber mechanism according to the invention.

FIG. 6A is a side view of a control arm of one embodiment of a grabber mechanism according to the invention.

FIG. 6B is a side view of a lower control finger of one embodiment of a grabber mechanism according to the invention.

FIG. 6C is a side view of an upper control finger of one embodiment of a grabber mechanism according to the invention.

FIG. 7 is a perspective view of one embodiment of a motor and grabber mechanism.

FIG. 8 is an exploded view of one embodiment of a grabber mechanism.

FIG. 9A is a top view of a back panel of one embodiment of a grabber mechanism according to the invention.

FIG. 9B is a perspective view of one embodiment of a back panel according to the invention.

FIG. 10A is a top view of a portion of one embodiment of a grabber mechanism according to the invention.

FIG. 10B is a perspective view of a portion of one embodiment of a grabber mechanism according to the invention.

FIG. 10C is a top view of a portion of one embodiment of a grabber mechanism according to the invention.

FIG. 10D is an end view of one embodiment of a grabber mechanism according to the invention.

FIG. 10E is a perspective view of embodiment of a grabber mechanism according to the invention.

FIG. 10F is a perspective view of embodiment of a grabber mechanism coupled to an elliptical tube according to the invention.

FIG. 11A is a perspective top view of one embodiment of a heparin pump according to the invention.

FIG. 11B is a perspective view of one embodiment of a heparin pump, without a housing, according to the invention.

FIG. 11C is a cut-away view of one embodiment of a heparin pump according to the invention.

### DETAILED DESCRIPTION OF THE INVENTION

While the present invention is capable of embodiments in various forms, as shown in the drawings, hereinafter will be described the presently preferred embodiments of the invention with the understanding that the present disclosure is to be considered as an exemplification of the invention, and it is not intended to limit the invention to the specific embodiments illustrated.

A hemodialysis system includes a dialyzer that is connected to both a blood flow path and a dialysate flow path. Both the blood flow path and dialysate flow path travel through the dialyzer to transport their respective fluids through closed loop systems wherein the dialysate flow path is isolated from the blood flow path by a semipermeable membrane. Preferably, the dialysate flows in the opposite direction to blood flow within the dialyzer, which possesses an inlet for receiving dialysate, an outlet for expelling dialysate, an inlet for receiving blood from a patient, and an outlet for returning blood to the patient. The blood flow path and dialysate flow path are conduits. The conduits may have an inside diameter of approximately 0.156 inch (3-5 millimeters). Both the blood flow path and the dialysate flow path pass through the dialyzer, but are separated by the dialyzer's semipermeable membrane. The dialyzer is of a construction and design known to those skilled in the art. Preferably, the dialyzer includes a large number of hollow fibers which form a semipermeable membrane. Suitable dialyzers can be obtained from Fresenius Medical Care, Baxter International, Inc., and Nipro Medical Corporation.

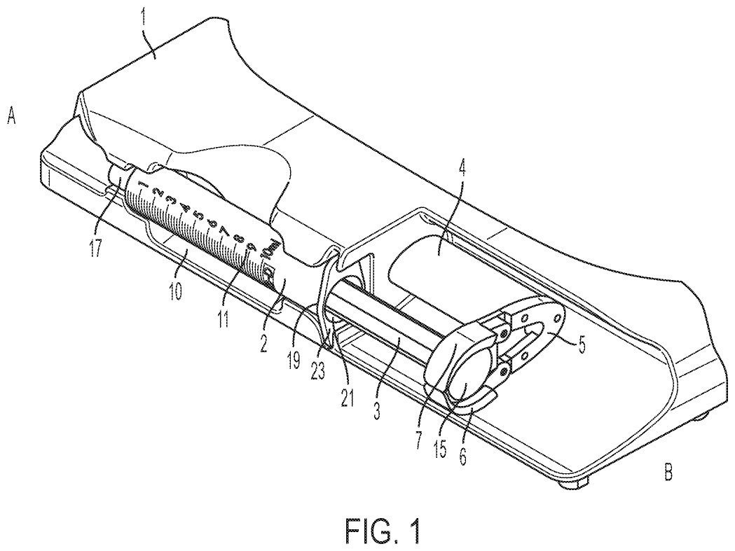

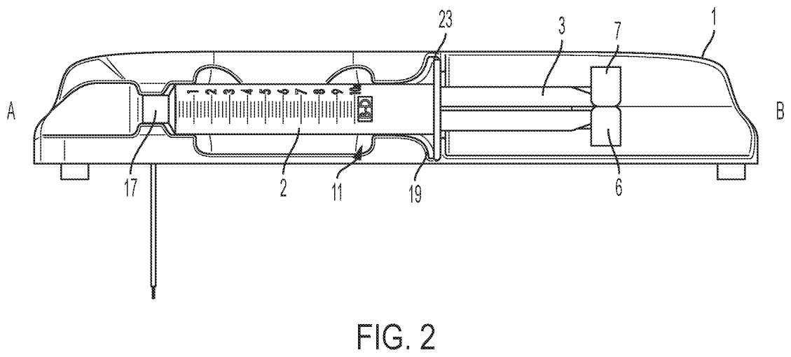

As seen in FIGS. 1-2, the syringe pump for delivery of a fluid, e.g., heparin, has an outer housing 1 that contains a recess 10 to house syringe 11, which includes syringe housing 2 and plunger 3. Plunger 3 is an elongate member having first end 13 and enlarged second end 15. Syringe housing 2 is an elongate tubular member having (e.g., a cylindrical barrel) having first end 17, second end 19, and a lumen 21 therebetween. First end 17 has an outlet or nose with an opening through which fluid, e.g., a medication such as heparin, can be dispensed. The second end has flange 23 and an opening, which communicates with lumen 21. The opening in the second end and lumen 21 of syringe housing 2 are sized to receive the elongate member of plunger 3. The nose or outlet at first end 17 is connected to an infusion line (not shown) through which the liquid can be dispensed by application of a force to plunger 3, thereby advancing plunger 3 towards first end 17 of the syringe barrel and end A of housing 1.

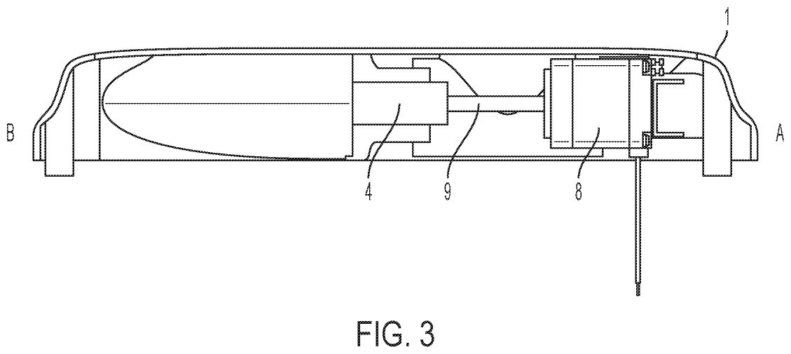

As seen in FIGS. 3, 4, and 7, the syringe pump has a drive mechanism that at least includes lead screw 9, stepper motor 8, and elliptical tube 4. Lead screw 9 is driven by stepper motor 8, which may be coupled to base plate 60 through motor mount 64. Elliptical tube 4 is configured to slide within a lumen of slide bushing 68. At least a portion of lead screw 9 passes through a lumen of elliptical tube or slide body 4 and couples to a female threaded element 62 located in the lumen of elliptical tube 4. Stop plate 66 keeps female threaded element 62 coupled to a first end of elliptical tube 4 and at least partially within the lumen of elliptical tube 4. A mechanical switch or an optical sensor (e.g., IR sensor 70) may be used to detect the presence of syringe 11. Grabber mechanism 120 is coupled to a second end of elliptical tube 4. The asymmetrical shape of elliptical tube 4 prevents the drive mechanism from rotating. Elliptical tube 4 is also easy to clean and seal.

The syringe pump also has a grabber mechanism coupled to the driver mechanism. Grabber mechanism 120 includes lower and upper control fingers 6, 7 that are connected to control arm 5 at connection points 25, 27. Control arm 5 is coupled to lead screw 9. Lower and upper control fingers 6, 7 are configured to engage enlarged end 15 of plunger 3. Movement of lead screw 9 by stepper motor 8 results in movement of elliptical tube 4, and therefore, movement of plunger 3. Thus, controlling the position of lead screw 9 using stepper motor 8 controls the dose of heparin. A separate encoder (either a linear encoder or a rotary encoder) may be used to independently verify that stepper motor 8 is turning lead screw 9 at an appropriate rate. A rotary encoder may be mounted to stepper motor 8 and provide feedback signals by tracking the speed and/or number of rotations of lead screw 9. A linear encoder includes a sensor that reads a scale and converts the encoded position into a signal that can be decoded into position. The encoders may be typical constructions known to those skilled in the art.

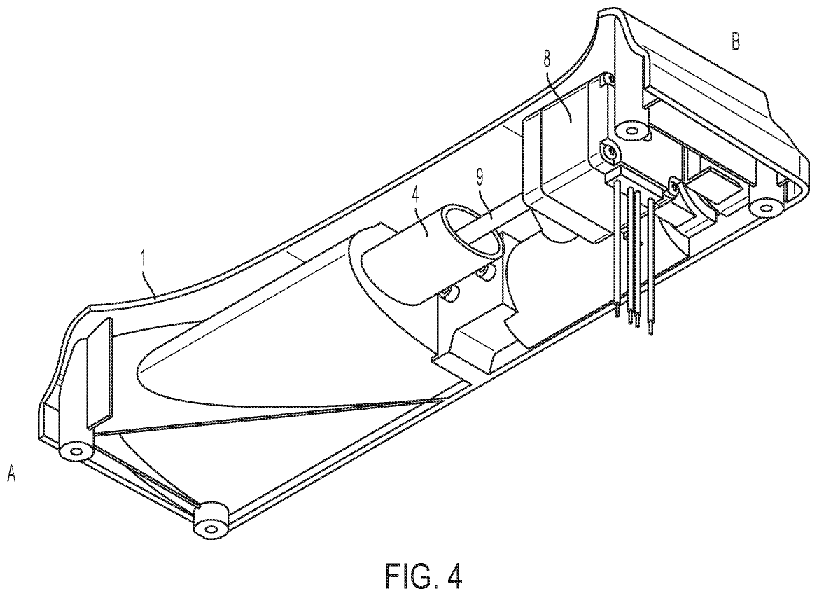

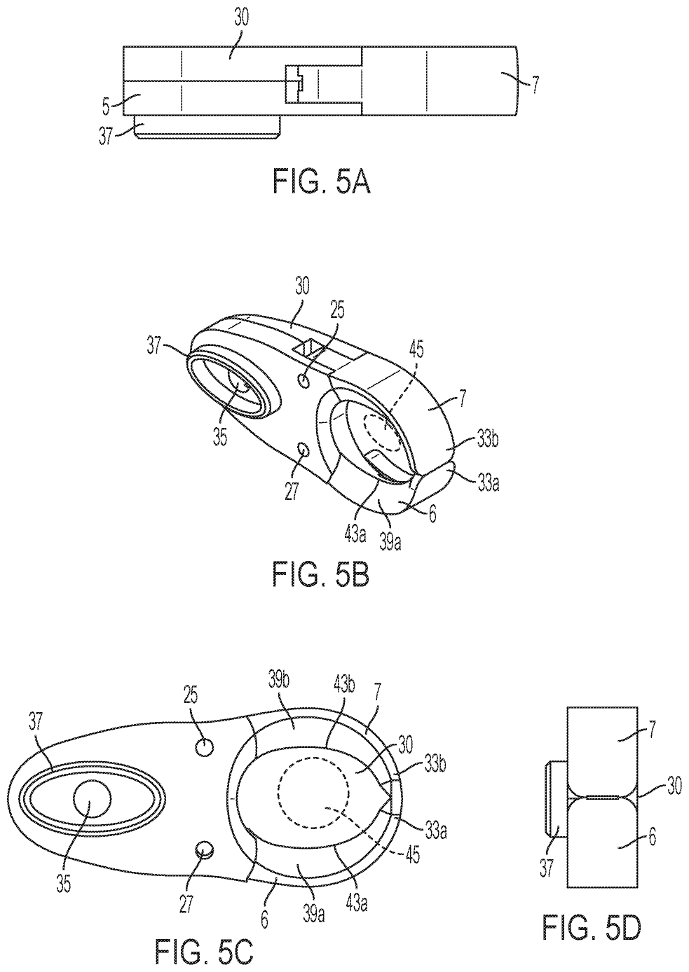

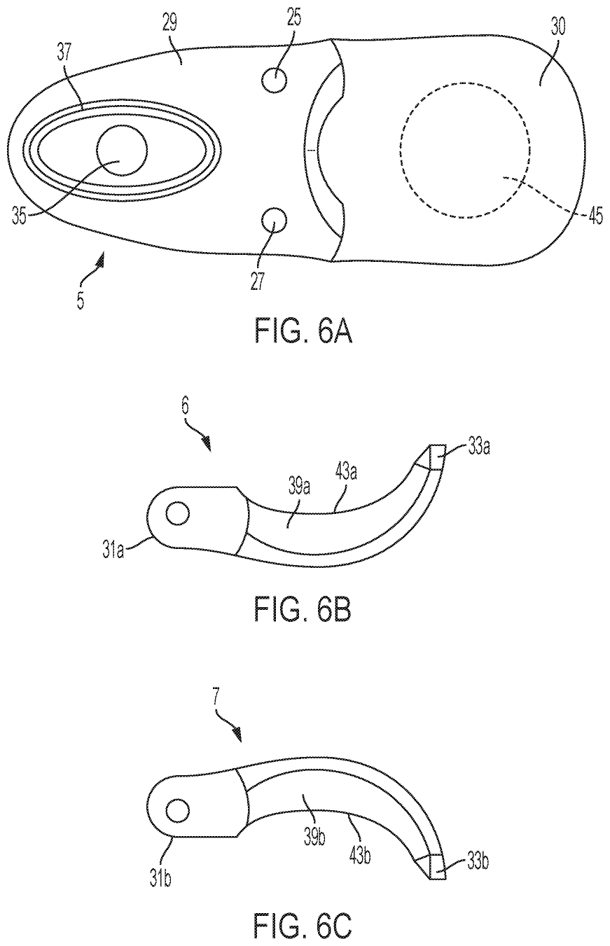

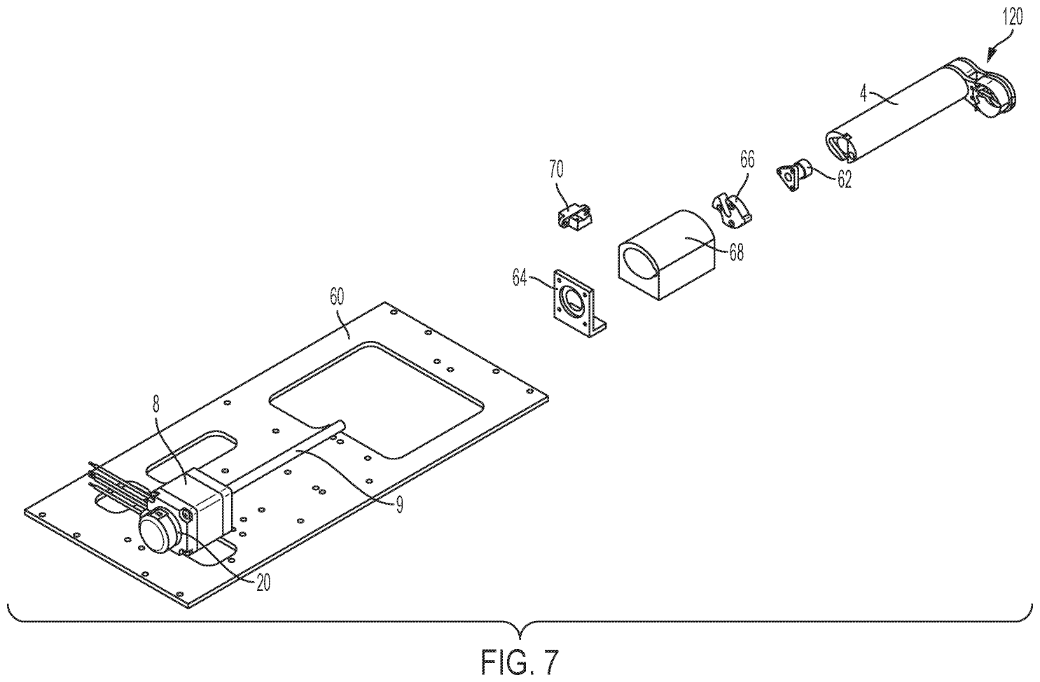

As seen in FIGS. 5A-D and 6A-C,the grabber mechanism 120 includes control arm or base plate 5, back panel or grabber body 30, and lower and upper control fingers 6, 7 that are configured to automatically grab enlarged end 15 of plunger 3. Control arm 5 has first and second connection points 25, 27 and body 29. Body 29 includes recess 35 configured to couple to lead screw 9 and elliptical tubular extension 39 configured to couple with elliptical tube 4. Lower and upper control fingers 6, 7 each have a first end 31a,b, a second end 33a,b, and a curved portion in between the first and second ends that is configured to grasp enlarged end 15 of plunger 3. Lower and upper control fingers 6, 7 also each have a beveled edge 39a,b along at least a portion of the curved portion with the thinnest edges 43a, located on the interior edge that forms an elliptical space between lower and upper control fingers 6, 7. Lower and upper control fingers 6, 7, e.g., lower control finger 6, include first and second portions. The first portion includes the beveled edge (as described above) and the second portion includes gap 41 formed by the opposite sides of the beveled surfaces and back panel 30. Gap 41 is configured to house enlarged end 15 of pusher 3. The first ends 31a,b of lower and upper control fingers 6,7 are connected to the first and connection points 25, 27 of control arm 5 via spring-loaded hinges. Control arm 5 and lower and upper control fingers 6, 7 form an elliptical space between the curved portions. The elliptical space enables control arm 5 and lower and upper control fingers 6, 7 to separate as they come into contact with enlarged end 15 of plunger 3. As detailed below, lower and upper control fingers 6, 7 can then automatically capture enlarged end 15 without any additional actions by the patient. Back panel 30 is connected to body 29, e.g., at connection points 25, 27, and extends beyond the edge of body 29 to at least cover the elliptical space formed between the curved portions of lower and upper control fingers.

The springs in the spring-loaded hinges are biased to keep second ends 33a,b of lower and upper control fingers 6, 7 in contact. As enlarged end 15 of plunger 3 comes into contact with lower and upper control fingers 6, 7, enlarged end 15 pushes against beveled edges 39a,b of lower and upper control fingers 6,7, thereby forcing the spring-loaded hinges to separate lower and upper control fingers 6, 7, thereby creating a wider space to accommodate enlarged end 15 of plunger 3. After enlarged end 15 pushes beyond inner edge 43a,b of beveled edges 39a,b, enlarged end 15 is no longer applying pressure to widen the opening between lower and upper control fingers 6,7 and lower and upper control fingers 6,7 once again close such that second ends 33a,b are in contact. In this closed position, enlarged end 15 of pusher 3 is sitting within gap 41 that is formed between the opposite sides of beveled edges 39a,b and back panel 30. With enlarged end 15 sitting in gap 41 and lower and upper control fingers 6,7 in the closed position, plunger 3 is temporarily coupled to the driver mechanism such that movement of lead screw 9 results in movement of plunger 3 through the syringe housing 2, resulting in dispensing fluid, such as heparin, out of the outlet of syringe 11.

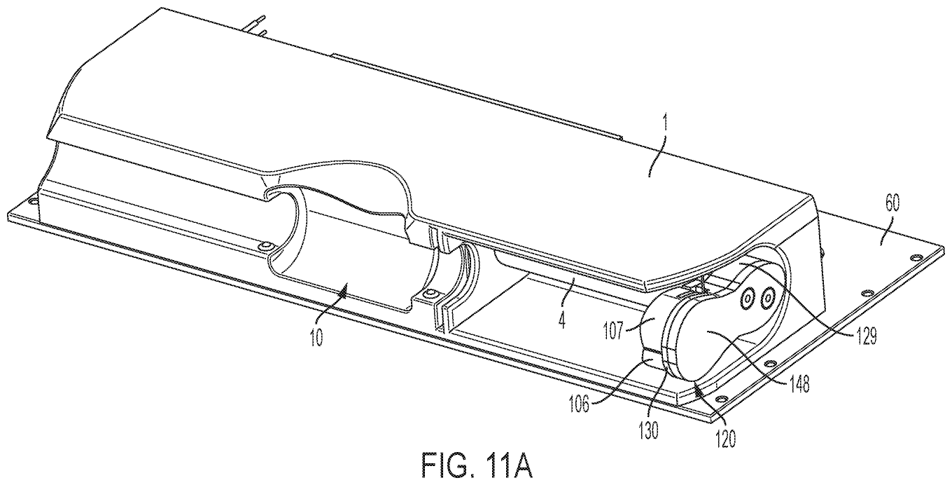

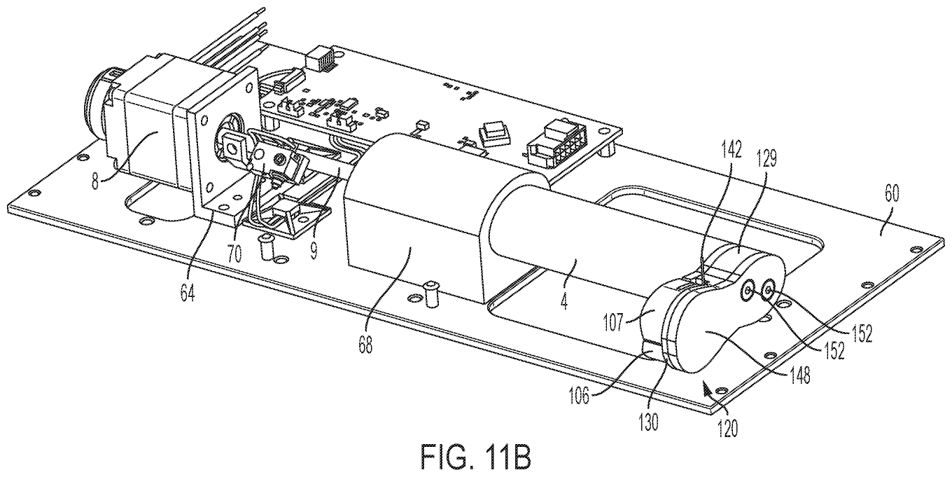

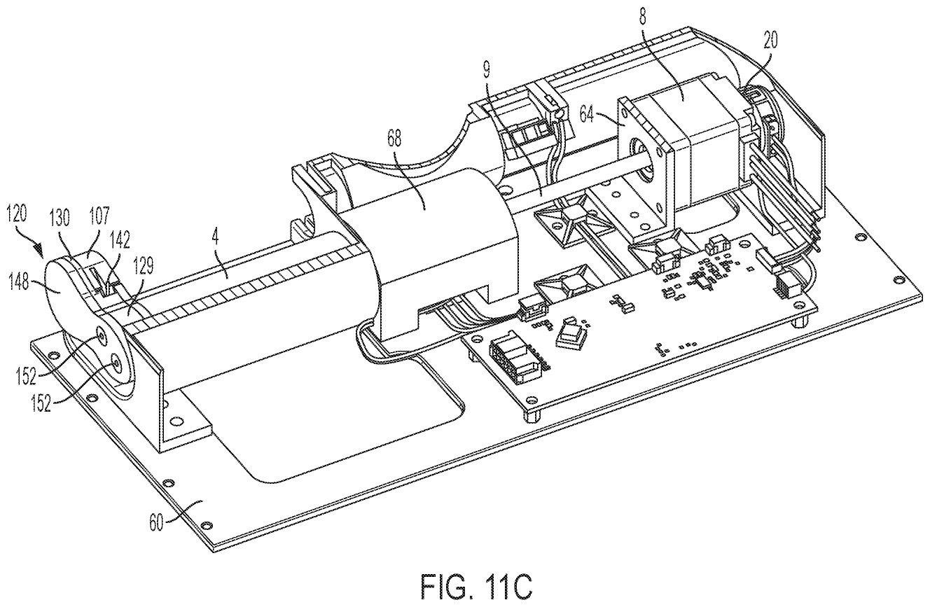

In an alternative embodiment, as seen in FIGS. 7-11C, a grabber mechanism may include a grabber base plate 105, lower and upper control fingers 106, 107, torsion springs 142, grabber body 129 with back panel 130, pressure plate 146, force sensor 145, and back cover 148. Grabber base plate 105 can be coupled to a second end of elliptical tube 4. Lower and upper control fingers 106, 107 are configured to engage enlarged end 15 of plunger 3, and fit between grabber base plate 105 and back panel 130 of grabber body 129. Dowel pins 140 and torsion springs 142 may couple lower and upper control fingers 106, 107 to base plate 105 and back panel 130, such that lower and upper control fingers 106, 107 are coupled through spring-loaded hinges.

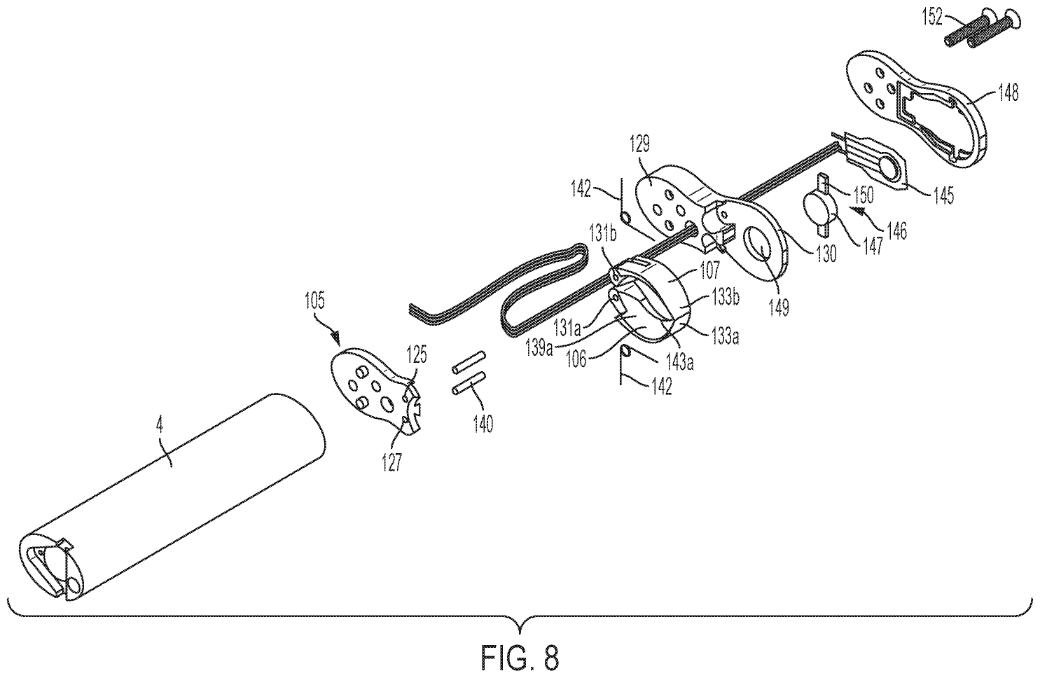

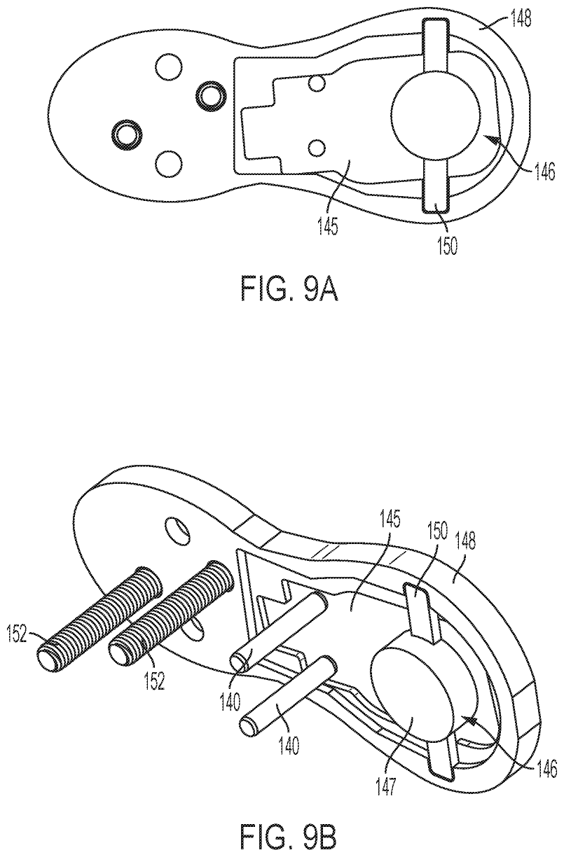

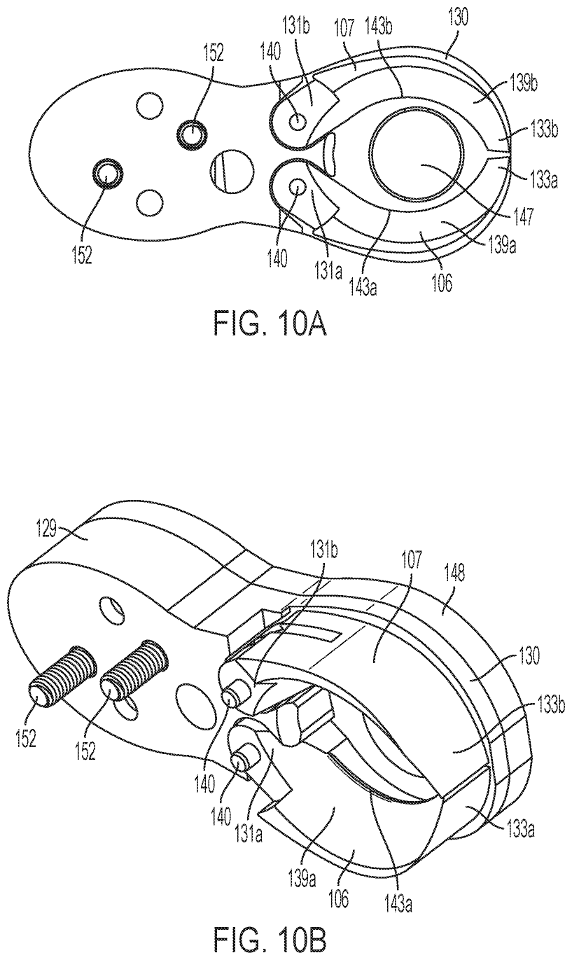

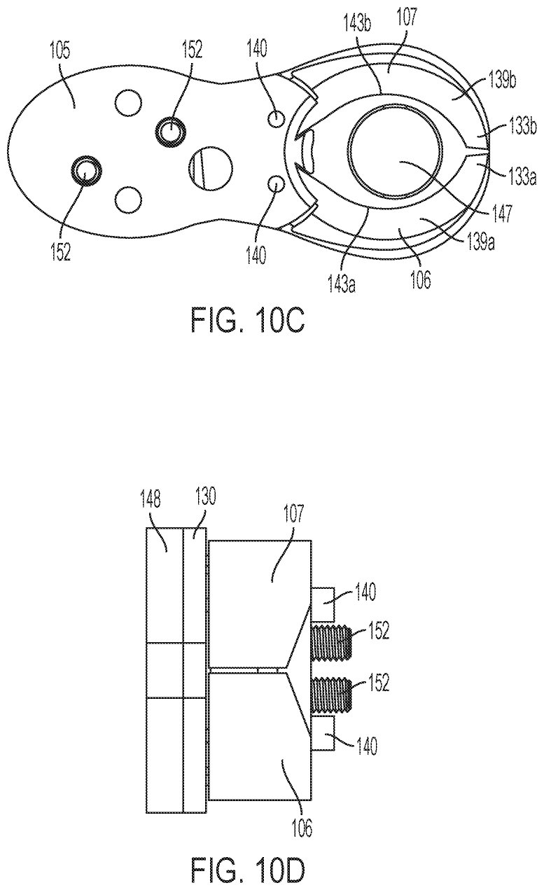

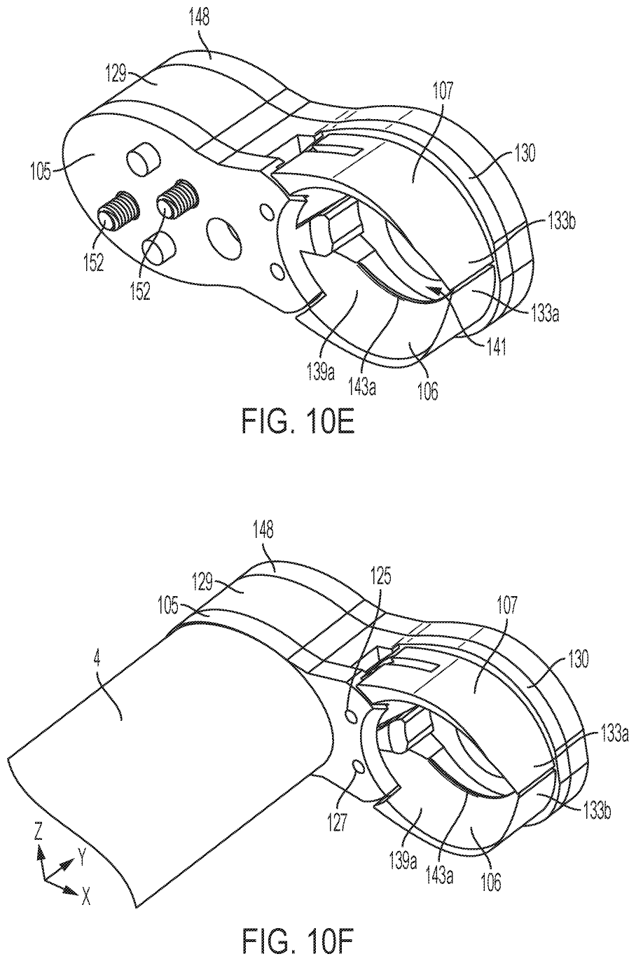

As seen in FIGS. 8 and 10A-10F, lower and upper control fingers 106, 107 are configured to automatically grab enlarged end 15 of plunger 3. Grabber base plate or control arm 105 has first and second connection points 125, 127 configured to be coupled with lower and upper control fingers 106, 107. Grabber base plate 105 may include a recess (not shown) configured to couple to lead screw 9 configured to couple with elliptical tube 4. Lower and upper control fingers 106, 107 each have a first end 131a,b, a second end 133a,b, and a curved portion in between the first and second ends that is configured to grasp enlarged end 15 of plunger 3. Lower and upper control fingers 106, 107 also each have a beveled edge 139a,b along at least a portion of the curved portion with the thinnest edges 143a,b located on the interior edge that forms an elliptical space between lower and upper control fingers 106, 107. Lower and upper control fingers 106, 107, include first and second portions. The first portion includes the beveled edge (as described above) and the second portion includes gap 141 formed by the opposite sides of the beveled surfaces and back panel 30. Gap 141 is configured to house enlarged end 15 of pusher 3. The first ends 131a,b of lower and upper control fingers 106, 107 are connected to the first and second connection points 125, 127 of grabber base plate 105 and back panel 130 via spring-loaded hinges. Lower and upper control fingers 106, 107 form an elliptical space between the curved portions. The elliptical space enables lower and upper control fingers 106, 107 to separate as they come into contact with enlarged end 15 of plunger 3. As detailed below, lower and upper control fingers 106, 107 can then automatically capture enlarged end 15 without any additional actions by the patient. Back panel 130 and grabber body 129 are connected to grabber base plate 105 and lower and upper control fingers 106, 107, e.g., at connection points 125, 127, through dowel pins 140 and torsion springs 142. Back panel 130 extends beyond the edge of body 129 to at least cover the elliptical space formed between the curved portions of lower and upper control fingers 106, 107. Back panel 130 includes a substantially circular opening 149 configured to fit at least a portion of pressure plate 146 therethrough. As seen in FIGS. 8 and 9A-9B, pressure plate 146 includes raised, substantially circular portion 147 and at least two extensions that extend therefrom. In operation, a first side of raised substantially circular portion 147 of pressure plate 146 extends through the circular opening 149 of back cover plate 148. A second side of raised substantially circular portion 147 contacts force sensor 145. Force sensor 145 is configured to sit within a recess of back cover plate 148. Screws 152 may be used to couple back cover plate 148, body 129, and base plate 105 together.

The springs in the spring-loaded hinges are biased to keep second ends 133a,b of lower and upper control fingers 106, 107 in contact. The spring-loaded hinges may include dowel pins 140 and torsion springs 142. As enlarged end 15 of plunger 3 comes into contact with lower and upper control fingers 106, 107, enlarged end 15 pushes against beveled edges 139a,b of lower and upper control fingers 106, 107, thereby forcing the spring-loaded hinges to separate lower and upper control fingers 106, 107, thereby creating a wider space to accommodate enlarged end 15 of plunger 3. After enlarged end 15 pushes beyond inner edge 143a,b of beveled edges 139a,b, enlarged end 15 is no longer applying pressure to widen the opening between lower and upper control fingers 106, 107 and lower and upper control fingers 106, 107 once again close such that second ends 133a,b are in contact. In this closed position, enlarged end 15 of pusher 3 is sitting within gap 41 that is formed between the opposite sides of beveled edges 139a,b and back panel 130. With enlarged end 15 sitting in gap 41 and lower and upper control fingers 106, 107 in the closed position, plunger 3 is temporarily coupled to the driver mechanism such that movement of lead screw 9 results in movement of plunger 3 through the syringe housing 2, resulting in dispensing fluid, such as heparin, out of the outlet of syringe 11.

The syringe pump may also contain sensors, such as force sensor 45, 145 and/or an optical sensor, and a processor (not shown) the analyzes the signals from the sensors. The processor could analyze the forces detected by sensor 45, 145. If force sensor 45, 145 registers that a higher force is necessary to advance enlarged end 15 of plunger 3 toward first end 17 of syringe 11, then the processor could detect an occlusion in the blood flow path. A detected force of greater than about 2 lbs, alternatively greater than about 3 lbs, alternatively greater than about 4 lbs, alternatively greater than about 5 lbs, alternatively greater than about 6 lbs may be indicative of an occlusion in the blood flow path. The processor could also detect when the plunger has been engaged by the grabber mechanism based on readings from force sensor 45, 145. A detected force of between about 1 lb to about 2 lbs may be indicative of engagement of the plunger by the grabber mechanism. If force sensor 45, 145 registers that a higher force is necessary to advance enlarged end 15 of plunger 3 toward first end 17 of syringe 11, then the processor could detect that the syringe is empty or near empty.

Force sensor 45, 145 may be a flexible resistive force sensor mounted on the grabber mechanism. Force sensor 45, 145 may be located in or on back panel 30 such that force sensor 45 will be adjacent the elliptical opening between the curved portions of lower and upper control fingers 6, 7. Thus, when enlarged end 15 is housed in gap 41, enlarged end 15 comes into contact with force sensor 45. Force sensor may be a typical force sensor known to those skilled in the art. The syringe pump may also include a light source and an optical sensor. The light source and optical sensor may be located on opposite sides of syringe 11 such that the processor may be able to determine the presence of a syringe in the housing recess 10 based on the signals detected from the optical sensor. The optical sensor may be a typical optical sensors known to those skilled in the art.

After the patient is finished dialyzing and no longer needs the heparin, the patient can simply pull syringe 11 free from housing 1. The grabber mechanism, which includes control arm 5 and lower and upper control fingers 6, 7, may automatically release plunger 3 when syringe 11 is pulled out of housing 1. Moreover, the design of housing 1 and the pump enable the patient both to load syringe 11 into housing 1 and pull syringe 11 free of housing 1 with only one hand, as is sometimes necessary during dialysis.

In many embodiments, a syringe pump configured to receive a syringe having a plunger movable within a lumen of an elongate tubular member is provided. The syringe pump includes a housing having a recess configured to receive at least a portion of the syringe; a drive mechanism for moving the plunger within the lumen, the drive mechanism comprising a motor and a lead screw; and a grabber mechanism, wherein the grabber mechanism comprises a control arm, a back panel, upper and lower control fingers, and a gap formed between the back panel and upper and lower control fingers, wherein the upper and lower control fingers each have first and second ends, and a curved portion therebetween having a width, an interior edge, and an exterior edge, wherein the first ends of the upper and lower control fingers are coupled to the control arm via first and second spring hinges, and wherein the gap is configured to house an enlarged end of the plunger, and wherein the control arm is coupled to the lead screw.

In some embodiments, the interior edges of the upper and lower control fingers form a substantially elliptical space therebetween when the second ends are in contact.

In some embodiments, at least a portion of each of the curved portions have a beveled surface such that the widths of the curved portions are smaller at the interior edges than at the exterior edges. In some embodiments, the gap is located between the back panel and an opposite side of each of the beveled surfaces of the upper and lower control fingers.

In some embodiments, the motor is a stepper motor.

In some embodiments, the drive mechanism further comprises an elliptical tube having a first end, a second end, a lumen therebetween, and a female threaded element configured to receive the lead screw. In some embodiments, the elliptical tube prevents rotation of the drive mechanism. In some embodiments, at least a portion of the lead screw is disposed within the lumen of the elliptical tube and coupled to the female threaded element. In some embodiments, the control arm further comprises an elongate elliptical extension and wherein the elliptical tube is coupled to the elongate elliptical extension.

In some embodiments, the grabber mechanism further comprises a force sensor. In some embodiments, the grabber mechanism further comprises a pressure plate, and wherein the pressure plate is configured to contact the force sensor and an enlarged end of a pusher of a syringe. In some embodiments, the force sensor is a flexible resistive force sensor. In some embodiments, the force sensor is capable of detecting an occlusion in a conduit connected to a syringe received in the syringe pump. In some embodiments, the force sensor is capable of detecting a presence of a plunger in the grabber mechanism.

In some embodiments, the syringe pump further comprises an optical sensor. In some embodiments, the optical sensor is located behind the syringe. In some embodiments, the optical sensor detects the presence of a syringe in the recess.

In some embodiments, the drive mechanism further comprises an encoder. In some embodiments, the encoder is configured to verify that the motor is turning the lead screw at a set rate. In some embodiments, the encoder is a linear encoder. In some embodiments, the encoder is a rotary encoder.

In many embodiments, method for infusing a medicament using a syringe pump is described. The method includes the steps of loading a syringe into a recess of a syringe pump, the syringe comprising an elongate tubular member having a first end and a second end, a plunger movable within a lumen of an elongate tubular member, and a medicament within the lumen, the syringe pump comprising a housing having the recess configured to receive at least a portion of the syringe, a drive mechanism for moving the plunger within the lumen, and a grabber mechanism, wherein the drive mechanism comprises a motor and a lead screw, wherein the grabber mechanism is coupled to the lead screw, and wherein the grabber mechanism engages an enlarged end of the plunger; and moving the plunger within the lumen of the elongate tubular member in a direction from the first end to the second end by operating the motor to move the lead screw and the grabber mechanism, wherein movement of the plunger in the direction from the first end to the second end causes the medicament to exit from the second end of the syringe.

In some embodiments, the grabber mechanism comprises a control arm, a back panel, upper and lower control fingers, and a gap formed between the back panel and upper and lower control fingers, wherein the upper and lower control fingers each have first and second ends, and a curved portion therebetween having a width, an interior edge, and an exterior edge, wherein the first ends of the upper and lower control fingers are coupled to the control arm via first and second spring hinges, and wherein the enlarged end of the plunger is housed within the gap. In some embodiments, at least a portion of each of the curved portions have a beveled surface such that the widths of the curved portions are smaller at the interior edges than at the exterior edges. In some embodiments, during the loading step, the enlarged end of the plunger pushes against the beveled surface of each of the curved portions, thereby forcing the first and second spring hinges to separate the upper and lower control fingers to accommodate the enlarged end of the plunger. In some embodiments, after the enlarged end of the plunger is no longer applying pressure to the beveled surface each of the curved portions, at least a portion of the upper and lower control fingers are in contact at a first end.

In some embodiments, the method further includes the step of removing the syringe after at least a portion of the medicament has been delivered from the second end.

In some embodiments, the drive mechanism further comprises an elliptical tube having a first end, a second end, a lumen therebetween, and a female threaded element configured to receive the lead screw. In some embodiments, the elliptical tube prevents rotation of the drive mechanism.

In some embodiments, the method further includes the step of sensing a force applied by the enlarged end with a force sensor associated with the grabber element. In some embodiments, the grabber mechanism comprises a back panel having a recess, wherein the force sensor is housed within the recess of the back panel. In some embodiments, the grabber mechanism further comprises a pressure plate adjacent the force sensor, wherein the enlarged end of the plunger contacts the pressure plate. In some embodiments, a force detected by the force sensor of greater than about 2 lbs is indicative of an occlusion in a conduit connected to the second end of the syringe. In some embodiments, a force detected by the force sensor of between about 1 lb to about 2 lbs is indicative of engagement of the enlarged end of the plunger by the grabber mechanism.

In some embodiments, the method further includes the step of detecting a presence of the syringe in the recess of the housing with an optical sensor.

In some embodiments, the method further includes the step of verifying that the motor is turning the lead screw at a set rate with an encoder. In some embodiments, the encoder is a linear encoder or a rotary encoder.

In some embodiments, a conduit connected to the second end of the syringe is part of a hemodialysis system.

In some embodiments, the medicament is heparin.

In closing, regarding the exemplary embodiments of the present invention as shown and described herein, it will be appreciated that a hemodialysis system is disclosed. The principles of the invention may be practiced in a number of configurations beyond those shown and described, so it is to be understood that the invention is not in any way limited by the exemplary embodiments, but is generally directed to a hemodialysis system and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention. Furthermore, the various features of each of the above-described embodiments may be combined in any logical manner and are intended to be included within the scope of the present invention.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the Specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present Specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

It should be understood that the processes, methods, and the order in which the respective elements of each method are performed are purely exemplary. Depending on the implementation, they may be performed in any order or in parallel, unless indicated otherwise in the present disclosure.

While several particular forms of the invention have been illustrated and described, it will be apparent that various modifications can be made without departing from the spirit and scope of the invention. Therefore, it is not intended that the invention be limited except by the following claims.

1. A syringe pump configured to receive a syringe having a plunger movable within a lumen of an elongate tubular member, the pump comprising:
a housing having a recess configured to receive at least a portion of the syringe;
a drive mechanism for moving the plunger within the lumen, the drive mechanism comprising a motor and a lead screw; and
a grabber mechanism, wherein the grabber mechanism comprises a control arm, a back panel, upper and lower control fingers, and a gap formed between the back panel and upper and lower control fingers, wherein the upper and lower control fingers each have first and second ends, and a curved portion therebetween having a width, an interior edge, and an exterior edge, wherein the first ends of the upper and lower control fingers are coupled to the control arm via first and second spring hinges, and wherein the gap is configured to house an enlarged end of the plunger, and
wherein the control arm is coupled to the lead screw.

2. The pump of claim 1, wherein the interior edges of the upper and lower control fingers form a substantially elliptical space therebetween when the second ends are in contact.

3. The pump of claim 1, wherein at least a portion of each of the curved portions have a beveled surface such that the widths of the curved portions are smaller at the interior edges than at the exterior edges.

4. The pump of claim 3, wherein the gap is located between the back panel and an opposite side of each of the beveled surfaces of the upper and lower control fingers.

5. The pump of claim 1, wherein the motor is a stepper motor.

6. The pump of claim 1, wherein the drive mechanism further comprises an elliptical tube having a first end, a second end, a lumen therebetween, and a female threaded element configured to receive the lead screw.

7. The pump of claim 6, wherein the elliptical tube prevents rotation of the drive mechanism.

8. The pump of claim 6, wherein at least a portion of the lead screw is disposed within the lumen of the elliptical tube and coupled to the female threaded element.

9. The pump of claim 6, wherein the control arm further comprises an elongate elliptical extension and wherein the elliptical tube is coupled to the elongate elliptical extension.

10. The pump of claim 1, wherein the grabber mechanism further comprises a force sensor.

11. The pump of claim 10, wherein the grabber mechanism further comprises a pressure plate, and wherein the pressure plate is configured to contact the force sensor and an enlarged end of a pusher of a syringe.

12. The pump of claim 10, wherein the force sensor is a flexible resistive force sensor.

13. The pump of claim 10, wherein the force sensor is capable of detecting an occlusion in a conduit connected to a syringe received in the syringe pump.

14. The pump of claim 10, wherein the force sensor is capable of detecting a presence of a plunger in the grabber mechanism.

15. The pump of claim 1, further comprising an optical sensor.

16. The pump of claim 15, wherein the optical sensor is located behind the syringe.

17. The pump of claim 15, wherein the optical sensor detects the presence of a syringe in the recess.

18. The pump of claim 1, wherein the drive mechanism further comprises an encoder.

19. The pump of claim 18, wherein the encoder is configured to verify that the motor is turning the lead screw at a set rate.

20. (canceled)

21. (canceled)

22. A method for infusing a medicament using a syringe pump, the method comprising:
loading a syringe into a recess of a syringe pump, the syringe comprising an elongate tubular member having a first end and a second end, a plunger movable within a lumen of an elongate tubular member, and a medicament within the lumen, the syringe pump comprising a housing having the recess configured to receive at least a portion of the syringe, a drive mechanism for moving the plunger within the lumen, and a grabber mechanism, wherein the drive mechanism comprises a motor and a lead screw, wherein the grabber mechanism is coupled to the lead screw, and wherein the grabber mechanism engages an enlarged end of the plunger; and
moving the plunger within the lumen of the elongate tubular member in a direction from the first end to the second end by operating the motor to move the lead screw and the grabber mechanism, wherein movement of the plunger in the direction from the first end to the second end causes the medicament to exit from the second end of the syringe.

23.-39. (canceled)
